(19) United States
(12) Patent Application Publication
(10) Pub. No.: US 2022/0378677 A1
(43) Pub. Date: Dec. 1, 2022

(54) TREATMENT OF TOOTH DECAY USING A CHEWING GUM COMPOSITION COMPRISING CANNABINOIDS

(71) Applicant: M. Mustix Ltd, Beit Shemesh (IL)

(72) Inventor: Doron STEINBERG, Jerusalem (IL)

(73) Assignee: M. Mustix Ltd, Beit Shemesh (IL)

(21) Appl. No.: 17/773,611

(22) PCT Filed: Nov. 1, 2020
(86) PCT No.: PCT/IL2020/051134

§ 371 Date: May 1, 2022

Related U.S. Application Data
(60) Provisional application No. 62/928,521, filed on Oct. 31, 2019.

(51) Int. Cl. A61K 8/42; A61Q 11/00; A61K 8/37; A61K 8/02

(52) U.S. Cl. CPC A61K 8/42; A61Q 11/00; A61K 8/375; A61K 8/0204; A61K 2800/92

(57) ABSTRACT

The invention is directed to a food product for use in the prevention, inhibition or treatment of tooth decay and other dental conditions, diseases and disorders, wherein said food product is a chewing gum comprising at least one cannabinoid.

### BACKGROUND OF THE INVENTION

Tooth decay, also known as dental caries or cavities, is a breakdown of teeth due to acids made by bacteria. The cavities may be a number of different colors from yellow to black. Symptoms may include pain and difficulty with eating. Complications may include inflammation of the tissue around the tooth, tooth loss, and infection or abscess formation.

The cause of cavities is acid from bacteria dissolving the hard tissues of the teeth (enamel, dentin and cementum). The acid is produced by the bacteria when they break down food debris or sugar on the tooth surface. Simple sugars in food are these bacteria's primary energy source and thus a diet high in simple sugar is a risk factor. If mineral breakdown is greater than build up from sources such as saliva, caries results. Risk factors include conditions that result in less saliva such as: diabetes mellitus, Sjogren's syndrome and some medications. Medications that decrease saliva production include antihistamines and antidepressants. Caries is also associated with poverty, poor cleaning of the mouth, and receding gums resulting in exposure of the roots of the teeth.

Prevention of dental caries includes regular cleaning of the teeth, a diet low in sugar, and small amounts of fluoride. Brushing the teeth twice per day and flossing between the teeth once a day is recommended. Fluoride may be acquired from water, salt or toothpaste among other sources. Treating a mother's dental caries may decrease the risk in her children by decreasing the numbers of certain bacteria she may spread to them. Screening can result in earlier detection. Depending on the extent of destruction, various treatments can be used to restore the tooth to proper function or the tooth may be removed. There is no known method to grow back large amounts of tooth. The availability of treatment is often poor in the developing world. Paracetamol (acetaminophen) or ibuprofen may be taken for pain.

Worldwide, approximately 3.6 billion people (48% of the population) have dental caries in their permanent teeth as of 2016. The World Health Organization estimates that nearly all adults have dental caries at some point in time. In baby teeth it affects about 620 million people or 9% of the population. They have become more common in both children and adults in recent years. The disease is most common in the developed world due to greater simple sugar consumption and less common in the developing world.

### SUMMARY OF THE INVENTION

The invention provides a food product comprising at least one cannabinoid, wherein said food product is a chewing gum; for use in the prevention, inhibition or treatment of tooth decay.

The invention further provides a food product comprising at least one cannabinoid, wherein said chewing gum; for use in the prevention, inhibition or treatment of a supra gingivae dental related condition disease or disorder.

When referring to a “food product” it should be understood to include any substance capable of being consumed by a mammal. Said food product can be of plant or animal origin. When referring to a chewable product it should be understood to encompass a product that can be chewed without being swallowed. A food product of the present invention is not a pharmaceutical composition.

When referring to a “chewing gum” it should be understood to include a soft, cohesive substance designed to be chewed without being swallowed. In some embodiments a chewing gum of the invention is composed of gum base, sweeteners, softeners/plasticizers, flavors, colors, and, typically, a hard or powdered polyol coating.

Chewing gum is primarily composed of a hydrophobic water-insoluble gum base and a water-soluble sugar or polyol phase. The water-insoluble part remains in the mouth during the whole mastication period, while the water-soluble substances dissolve and are ingested.

Chewing gum of the invention are solid or semi-solid, single-dose preparations with a base consisting mainly of masticatory gums that are intended to be chewed but not swallowed. It is a delivery system that contains one or more active ingredients (at least one cannabinoid) which are released by chewing. After dissolution or dispersion of the active ingredient(s) in saliva, these ingredients are released from the chewing gum within the oral cavity can act locally, be absorbed via the buccal mucosa. In some embodiments said chewing gum may be a sugar free-gum, a bubble gum, a palette gum, a center filled gum, a stick gum, a ribbon gum, a tab gum, a tube gum, a dragée gum and any combination thereof.

Typical ingredients of chewing gum may include gum base (main ingredients of the. gum base include resin, wax, and elastomer. Resin (ex. terpene) is the main chewable portion. Wax softens the gum. Elastomers add flexibility), one or more sweeteners (including sugar alcohols and/or artificial sweeteners: bulk polyol sweeteners are responsible for initial sweetness, whereas intensive sweeteners are intended for prolonging the sweetness effect. Intensive Sweeteners are often encapsulated to delay the release of flavor. Bulk Polyol Sweetener: include sugar, dextrose, glucose or corn syrup, erythritol, isomalt, xylitol, maltitol, mannitol, sorbitol, lactitol. Intensive Sweeteners include: aspartame, acesulfame-K, saccharine, sucralose, neohesperidine, dihydrichalcone), Glycerine (used to maintain moistness), one or more softener/plasticizer (sued to soften gum by increasing flexibility and reducing brittleness by altering the glass transition temperature and include lecithin, hydrogenated vegetable oils, glycerol ester, lanolin, methyl ester, pentaerythritol ester, rice bran wax, stearic acid, sodium and potassium stearates), one or more flavoring agents (used for taste and sensory appeal. Flavor components in gum exist in liquid, powder or micro-encapsulated forms. Liquid flavor incorporations are either water-soluble, oil-soluble, or water-dispersible emulsions. The oil-soluble flavors remain in the gum longer, resulting in longer lasting flavor sensations, because the gum base is hydrophobic and attracted to oil-based components. Flavoring agents may be natural or synthetic and include peppermint and spearmint, food acids are implemented to provide a sour flavor (i.e. citric, tartaric, malic, lactic, adipic, and fumaric acids), one or more coloring agents, one or more coating agents (for pellet gum's characteristic hard outer shell is due to a polyol coating. Polyols can also be implemented as a water absorbent powder dusting in order to maintain the quality and extend the shelf life of the product. These humectants bind water by establishing many hydrogen bonds with water molecules. Such coatings include sorbitol, maltitol/isomalt, mannitol, starch).

In some embodiments, said chewing gum is a sugar free chewing gum. In some embodiments a product of the present invention comprises sorbitol in the range of between about 10-70%. In some embodiments a product of the present invention comprises GUM BASE (polaris-t) in the range of between about 15-50%. In some embodiments a product of the present invention comprises GUM BASE (GEMINIS) in the range of between about 15-50%. In some embodiments a product of the present invention comprises syrup maltitol in the range of between about 5-20%. In some embodiments a product of the present invention comprises lecithin in the range of between about 0.1-2%. In some embodiments a product of the present invention comprises glycerin in the range of between about 0.2-2%. In some embodiments a product of the present invention comprises calcium carbonate in the range of between about 2-20%. In some embodiments a product of the present invention comprises mannitol in the range of between about 2-20%. In some embodiments a product of the present invention comprises powder flavor in the range of between about 0.1-5%. In some embodiments a product of the present invention comprises liquid flavor in the range of between about 0.1-5%. In some embodiments a product of the present invention comprises crystal mint in the range of between about 0.01-2%. In some embodiments a product of the present invention comprises sucralose in the range of between about 0.1-2%.

A food product of the present invention further comprises at least one ingredient selected from a breath freshener, a flavoring agent, a colorant, an anti-microbial agent, an anti-inflammatory agent, teeth brightening agent, teeth strengthening agent, agent capable of dilating the upper airways and any combinations thereof.

In some embodiments, said at least one cannabinoid compound is an endo-cannabinoid compound.

In further embodiments, said at least one cannabinoid compound is select from cannabinoid or endocannabinoid derivatives or precursors or synthetic derivatives as: ARAS (arachidonoyl serine), 2AG (2-arachidonoyl glycerol), AEA (arachidonoyl ethanolamide), OEA (oleoyl ethanolamide), OG (oleoyl glycine), OA (oleoyl alanine), HU-210, HU-308, PEA (palmitoyl ethanolamide) HU-433, AraG (Arachidonoyl glycine), PG (Palmitoyl glycine), AraA (Arachidonoyl alanine), PA (Palmitoyl alanine), PS (Palmitoyl serine), OS (Oleoyl serine), 2-arachidonoyl glyceryl ether, 2-oleoyl glyceryl ether , 2-palmitoyl glyceryl ether and any derivative or combinations thereof.

When referring to a “cannabinoid compound” it should be understood to encompass any compound that acts on cannabinoid receptors and or the endocannabinoid pathway or part of the biosynthesis of those compounds. Such compounds include but are not limited to endocannabinoids (produced naturally in the body by animals), phytocannabinoids (found in some plants), synthetic and semi-synthetic cannabinoids (manufactured artificially). In some embodiments, said at least one cannabinoid compound is an endo-cannabinoid compound.

In some embodiments said cannabinoid compound is added to said chewing gum in the form of an oil. In other embodiments, said cannabinoid compound is added to said chewing gum in the form of nano size particulate matter.

In other embodiments, said at least one cannabinoid compound is select from ARAS (arachidonoyl serine), 2AG (2-arachidonoyl glycerol), AEA (arachidonoyl ethanolamide), OEA (oleoyl ethanolamide), OG (oleoyl glycine), OA (oleoyl alanine), HU-210 (1,1-Dimethylheptyl-11-hydroxy-tetrahydrocannabinol), HU-308 ([(1R,2R,5R)-2-[2,6-dimethoxy-4-(2-methyloctan-2-yl)phenyl]-7,7-dimethyl-4-bicyclo[3 .1.1]hept-3-enyl]methanol)), PEA (palmitoyl ethanolamide) HU-433 ([(1R,5R)-2-[2,6-dimethoxy-4-(2-methyloctan-2-yl)phenyl]-6,6-dimethyl-4-bicyclo[3.1.1]hept-3-enyl]methanol), AraG (Arachidonoyl glycine), PG (Palmitoyl glycine), AraA (Arachidonoyl alanine), PA (Palmitoyl alanine), PS (Palmitoyl serine), OS (Oleoyl serine), 2-arachidonoyl glyceryl ether, 2-oleoyl glyceryl ether, 2-palmitoyl glyceryl ether and any derivative or combinations thereof.

In some embodiments, said at least one cannabinoid comprises at least 0.5% of the food product. In other embodiments, said at least one cannabinoid comprises at least 5% of the food product. In some embodiments, said at least one cannabinoid comprises at least 10% of the food product. In some embodiments, said at least one cannabinoid comprises at least 20% of the food product. In some embodiments, said at least one cannabinoid comprises at least 30% of the food product. In some embodiments, said at least one cannabinoid comprises at least 40% of the food product. In some embodiments, said at least one cannabinoid comprises at least 50% of the food product. In some embodiments, said at least one cannabinoid comprises at least 60% of the food product. In some embodiments, said at least one cannabinoid comprises at least 70% of the food product. In some embodiments, said at least one cannabinoid comprises at least 80% of the food product. In some embodiments, said at least one cannabinoid comprises at least 90% of the food product.

In some embodiments, said at least one cannabinoid comprises between about 0.5 to 10% of the food product. In some embodiments, said at least one cannabinoid comprises between about 1 to 15% of the food product. In some embodiments, said at least one cannabinoid comprises between about 5 to 25% of the food product. In some embodiments, said at least one cannabinoid comprises between about 10 to 50% of the food product. In some embodiments, said at least one cannabinoid comprises between about 20 to 60% of the food product.

In some embodiments, said at least one cannabinoid is in the form of an oil. In other embodiments, said at least one cannabinoid is in the form of a powder. In some embodiments, said at least one cannabinoid is homogeneously spread in the food product. In other embodiments, said at least one cannabinoid is concentrated at the core of said food product.

As used herein, the term “gum base” generally refers to a chewable rubber or plastic, which provides the structural integrity of the chewing gum. It may comprise either natural materials (such as the plant resin chicle) or synthetic materials (such as, for example, paraffin wax and related polymers). The gum base may further comprise a combination of elastomers, resins, waxes, fats, emulsifiers, fillers, or antioxidants as desirable to modify the texture of the chewing gum.

As used herein, the term “fixing agent” generally refers to a polyol that allows the gum to have a hard-outer coating. The polyol may be Sorbitol, Maltitol/Isomalt, Mannitol, Starch, and the like.

As used herein, the terms “sweeteners” and “flavors” generally refer to compounds added in order to improve the taste of the chewing gum. It is contemplated that various common sweeteners, such as xylitol and stevia, may be used to enhance the taste of the chewing gum. Other sweeteners used may include sugar, dextrose, glucose or corn syrup, erythritol, isomalt, maltitol, mannitol, sorbitol, lactitol, aspartame, acesulfame-K, saccharine, sucralose, neohesperidine, dihydrichalcone. Other flavors used may be natural or synthetic and may be peppermint, spearmint, or a sour acid, such as citric, tartaric, malic, lactic, adipic, and fumaric.

As used herein, the term “additives” generally refers to non-structural or flavor-related compounds that are added to the chewing gum to impart other desirable properties. These may include various vitamins for imparting healthful effects to the user. Caffeine and gingerol are two additives that might be used.

The gum of the present disclosure may also have a softener or plasticizer, such as lecithin, hydrogenated vegetable oils, glycerol ester, lanolin, methyl ester, pentaerythritol ester, rice bran wax, stearic acid, sodium potassium stearates, and the like.

The chewing gum may further comprise gingerol, caffeine, flavorings, and sweeteners, preferably artificial sweeteners. In some embodiments, the addition of vitamins or antibiotics may provide added benefits to users.

In some embodiments, a chewing gum of the invnetion may be produced by the following steps: Providing a gum base, CBD oil, mint oil, stevia, and a fixing agent; Adding the gum base to a mixer; Mixing the gum base for a duration sufficient to convert the gum base from individual pellets or balls into a semi-consistent, rubbery solid; Adding CBD, mint oil, stevia, caffeine and/or gingerol, and fixing agent to the mixture; Stirring the CBD, mint oil, stevia, caffeine and/or gingerol, fixing agent, and gum base together until a homogeneous rubbery solid develops; Transferring said homogeneous rubbery solid to a conveyer belt; Feeing said homogeneous rubbery solid through a compressing bottleneck in said conveyer belt, such that the resulting gum mixture is pressed into thin sheets; and Mechanically separating the thin gum sheets into small pieces fit for easy human chewing and use.

When referring to a “dental related condition, disease or disorder” it should be understood to encompass any type of condition, disease or disorder that is affecting the supra-gingivae area; teeth, gum, inner mouth tissue, tongue and any combinations thereof. Such dental related condition, disease or disorder may be caused by age related conditions, osteoporosis, poor dental hygiene (including low dental care utilization and poor oral hygiene), overly aggressive oral hygiene, immune related conditions, cancer (including side effects of cancer treatment), medications and conditions that dry the mouth, smoking, trauma, genetic factors, stress, mental health issues such as depression, pre-existing conditions such as diabetes and so forth.

Dental caries (tooth decay) relates to a condition resulting from microbial biofilm (plaque) formed on the tooth surface converts the free sugars contained in foods and drinks into acids that dissolve tooth enamel and dentine over time. With continued high intake of free sugars, inadequate exposure to fluoride and without regular microbial biofilm removable, tooth structures are destroyed, resulting in development of cavities and pain, impacts on oral-health-related quality of life, and, in the advanced stage, tooth loss and systemic infection.

Tooth loss is mainly caused by dental caries and periodontal diseases, leading up to edentulism. Severe tooth loss and edentulism (no natural teeth remaining) are widespread and particularly seen among older people.

In some embodiments, said dental related condition, disease or disorder is selected from tooth decay, dental plaque formation, periodontal disease, gingivitis, supra-gingivae disease, halitosis and any combinations thereof.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The Anti-Biofilm/Antimicrobial Effects of CBD-Chewing Gum of the Invention

A chewing gum is immersed in a 20 ml artificial saliva fluid for 15 min. while being in a pestle and mortar at a rate of one crush for 1 sec.

Artificial saliva samples are taken every 3 min, while supplementing the media with new fresh artificial saliva. The samples are placed with different oral bacteria as Streptococcus mutant, Streptococcus sobrinus (highly cariogenic bacteria) and incubated at 37C for a period of 24 hours.

The antimicrobial effect is examined by OD/CFU/qPCR. The anti-biofilm effect will be tested while incubating the artificial saliva with 2% sucrose. The biofilm is examined by CV/MTT and CLSM. The chewing gums is tested against placebo chewing gums. The effect of the chewing gum to affect microbial growth and biofilm is tested also when the strip of the chewing gum is placed in a test tube with and without sucrose.

Comparison of a Chewing Gum of the Invention with a Sugar-Less Chewing Gum CBD—chewing gum of the invention is compared to sugarless chewing gum of Orbit, Wrigley.

The chewing gums are placed in a media of artificial saliva and pressure at a rate of 1 sec applied for a period of 15 min-mimicking chewing conditions in the mouth.

Supernatant fluid is taken every 2 min and placed in a suspension of cariogenic bacteria as streptococcus mutans—the effect on the growth of the bacteria is measured for 24 hours.

The chewing process above is conducted on hydroxyapatite tables (mimicking the tooth) covered with cariogenic bacteria biofilm (mimicking the dental plaque) and the amount of bacteria on the hydroxyapatite and in the artificial saliva is measured every 2 min

The results of the CFU/OD/MTT/SEM/CLSM are compared between the CBD-chewing gum of the invention and the reference chewing gum and placebo chewing gum.

1. A method of preventing, inhibiting or treating tooth decay and/or dental related condition, disease or disorder in a subject in need thereof; said method comprises administering to said subject a food product comprising at least one cannabinoid, wherein said food product is a chewing gum product; for use in the prevention, inhibition or treatment of tooth decay.

2. (canceled)

3. A method according to claim 1, wherein said at least one cannabinoid compound is an endo-cannabinoid compound or derivative or precursor,

4. A a method according to claim 1, wherein said at least one endo cannabinoid derived compound is select from RAS (arachidonoyl serine), 2AG (2-arachidonoyl glycerol), AEA (arachidonoyl ethanol amide), OEA (oleoyl ethanolamide), OG (oleoyl glycine), OA (oleoyl alanine), FIV-308, PIHA (pahnitoyl ethanolamide) AraG (Arachidonoyl glycine), PG (Palmitoyl glycine), AraA (Arachidonoyl alanine), PA (Palmitoyl alanine), PS (Palmitoyl serine), OS (Oleoyl serine), 2-arachidonoyl glyceryl ether, 2-oleoyl glyceryl ether , 2-palmitoyl glyceryl ether and any derivative or combinations thereof.

5. A method according to claim 1, wherein said food product comprises synthetic, plant derived or derivatives of cannabinoid or endo cannabinoids.

6. A method according to claim 1, wherein said food product further comprises at least one ingredient selected from a breath freshener, a flavoring agent, a colorant, an anti-microbial agent, an anti-inflammatory agent, teeth brightening agent, teeth strengthening agent, agent capable of dilating the upper airways and any combinations thereof.

7. A method according to claim 1, wherein said dental related condition disease or disorder is selected from tooth decay, dental plaque formation, supra-gingivae diseases, halitosis and any combinations thereof.

8. (canceled)

9. (canceled)
